(19) United States
(12) Patent Application Publication
(10) Pub. No.: US 2006/0182737 A1
(43) Pub. Date: Aug. 17, 2006

(54) Wet wipes including a composition for reduction and prevention of wrinkles on the skin

(76) Inventor: Maria Kathleen Cruse, El Cajon, CA (US)

Correspondence Address: GORDON & REES LLP, 101 WEST BROADWAY, SUITE 1600, SAN DIEGO, CA, 92101, US

(21) Appl. No.: 11/260,349

(22) Filed: Oct. 27, 2005

Related U.S. Application Data
(63) Continuation of application No. 11/056,496, filed on Feb. 11, 2005.

(51) Int. Cl. A61K 38/46; A61K 9/70

(52) U.S. Cl. 424/94.65; 424/443

(57) ABSTRACT

Wet wipes comprising a composition for reducing and preventing wrinkles on the skin is disclosed. The composition is provided in the form of an aqueous composition comprising containing papain, urea, boric acid, preservatives, the sodium salt of 2-pyrrolidone-5-carboxylic acid, and almond extract.

This application is a continuation-in-part of U.S. patent application Ser. No. 11/056,496, filed Feb. 11, 2005.

### FIELD OF THE INVENTION

The invention relates to a composition for dermatological or cosmetic use for reducing wrinkles and lines on the skin. A method for treating skin to reduce wrinkles and lines on the skin includes topically applying the composition.

### BACKGROUND OF THE INVENTION

Skin is damaged by many extrinsic (environmental) and intrinsic factors (aging). Whether extrinsic or intrinsic, the damage results in wrinkling of the skin. As the skin naturally ages, there is a reduction in the cells and blood vessels that supply the skin. There is also a flattening of the dermal-epidermal junction which results in weaker mechanical resistance of this junction. Oikarinen, A., 1990, The Aging of Skin: Chronoaging Versus Photoaging, Photodermatal. Photoimmunol. Photomed. 7:34. At the molecular level, these changes have been correlated with biochemical changes in the content and structure of the extracellular matrix to which the major cells of the dermis (i.e., the fibroblasts) reside. Collagen becomes highly cross-linked and inelastic, elastin is reduced in amounts and is incorrectly distributed, and the glycosaminoglycans become reduced in amounts, which results in reduced intercellular water and leads to the formation of wrinkles.

For many people, wrinkles are a reminder of the disappearance of youth. Many efforts have been undertaken to reduce and eliminate wrinkles, resulting in a booming business in youth-conscious societies. Treatments range from non-invasive topical cosmetic creams and moisturizers to invasive measures such as collagen injections, tissue resurfacing and cosmetic surgery.

The problem of alleviating wrinkles and improving the health and appearance of wrinkled skin is ubiquitous and cosmetic methods for treatment of wrinkles are avidly sought.

Invasive measures for elimination of wrinkles includes cosmetic tissue resurfacing, in which a laser ablates a thin epidermal layer of a patient. During healing, a new epidermal layer is formed on the ablated surface, eliminating the previously existing wrinkles. However, a disadvantage of cosmetic tissue resurfacing is that the exact circumference of the surface tissue area to be treated is defined manually by the operator. Manual control easily results in accidental damage to healthy tissue due to involuntary movements of the operator's hand.

Components of topical cosmetic compositions include physiologically active materials obtained from animals, plants and microorganisms. One cosmetic composition used to treat wrinkles contains an extract of a bacterium from the family Pseudomonadaceae (U.S. Pat No. 6,806,070). Other compositions use exfoliating enzymes in conjunction with botanicals (U.S. Pat. No. 6,416,769), or contain hydrophilic particles to peel and remove dead skin cells (U.S. Pat. No. 6,808,715). These agents are used to clean the corneum stratum (the outer most layer of the epidermis) by loosening the bonds that hold the dead skin cells together, giving a smooth appearance to the skin. However, this result is only temporary, and the desired effects of these compositions may not be quickly perceived, leading to customer dissatisfaction. Thus, there is still a need for skin care compositions that rapidly and effectively treat wrinkles and fine lines, and restore the youthful appearance of the skin.

A wrinkle, as used herein, is a configurational change in the skin's surface. Burke, K. E., 1990, Facial Wrinkles, Postgraduate Medicine 88(1):207-227. Wrinkles can be classified into three classes: linear wrinkles, glyphic wrinkles, and crinkles. Kligman, A. M., et al., 1985, The Anatomy and Pathogenesis of Wrinkles, Br. J. Derm. 113:3742. Linear wrinkles are straight, found generally in the facial skin, and are caused by chronological and environmental factors. Glyphic wrinkles are shaped as triangles or rectangles of wrinkles, are found on the face, hands, and neck. Glyphic wrinkles are caused by exposure to sunlight, and are aggravated by exposure to ultraviolet light. Crinkles are thin, crinkled wrinkles on flabby skin, found anywhere on the skin, but typically on the backs of hands and around the eyelids. Wrinkles include, and are sometimes referred to as, lines, fine wrinkles, crinkles, crow's feet, or sags.

Proteolytic enzymes have been used in topical therapy for such things as scar removal from burn wounds and as an adjunct to anti-microbial therapy. Greenberg and Winnick, 1941, Plant Proteases, J. Biol. Sci. 135:761-787; Rodeheaver, G., et al., 1975, Abstract Proteolytic Enzymes as Adjuncts to Antimicrobial Prophylaxis of Contaminated Wounds, Am. J. Surg. 129(5):537-544. These enzymes include those generally restricted to plant sources, such as papaya (papain), fig (ficin), and pineapple (bromelain). Glazer and Smith, 1971, Papain and Other Plant Sulfhydryl Proteolytic Enzymes, in The Enzymes, P. Boyer, ed., Academic Press, N.Y., 3:501-546. Papain is found in papaya, and is at the peak of its activity in unripe fruit. Uses of papain include: tenderizing meat, defibrinating wounds, clotting milk, shrink proofing of wool, reducing viscosity and increasing palatability of pet food, preventing cornea scar deformation, treating jellyfish and insect sings, treating edemas, reducing inflammatory processes, as an ingredient in cleaning solutions for soft contact lenses, and also as an indigestion medicine. U.S. Pat. No. 5,441,740 to Ozlen discloses a cosmetic composition containing at least one alpha-hydroxy acid, salicylic acid, and at least one enzyme derived from fruit; preferable the digestive enzyme is a mixture of bromelain and papain. The composition is allegedly useful for treating various cosmetic conditions such as lack of adequate skin firmness, wrinkles, and dry skin. Papain has also been used in moisturizing creams and lotions and in exfoliating cleansers. Papain has also been used as an ingredient in anti-wrinkle and firming creams; however, the enzymatic activity of papain is short-lived in a cream.

### SUMMARY OF THE INVENTION

The present invention is directed to an aqueous carbohydrate-based topical gel polymer composition comprising a hydrolase and method of treatment for removing and preventing skin wrinkles. In a preferred embodiment, the composition may also contain papain as the proteolytic enzyme. The composition may also contain urea, boric acid, preservatives, the sodium salt of 2-pyrrolidone-5-carboxylic acid (NaCPA), and almond extract.

The carrier according to this invention should contain at least one gelling agent known in the art. A carbohydrate-based polymer is used as a gelling agent for the composition. In a preferred embodiment, the gel polymer is xanthan gum, sold under the name AMAZE XT™ (National Starch & Chemical).

The present invention further provides a method for administering hydrolases in a gel carrier for dermatological use. The composition is topically, regularly applied to whatever skin areas require treatment, i.e. involved areas, with the frequency and in the amount necessary to achieve the desired results. The gel composition may be used several times a day, several times a week, depending on the age and condition of the skin. Preferably, the composition is applied at least once per day, most preferably twice or three times per day, five to seven days per week.

### DETAILED DESCRIPTION OF THE INVENTION

A composition and method is provided for removing and preventing wrinkles, which comprises a hydrolase and a carbohydrate based gel.

Hydrolases

A hydrolase is defined as an enzyme that catalyzes the hydrolysis of various chemical bonds. Hydrolases such as carboxyesterase, triacylglycerol lipase, esterase, lipoprotein lipase, or proteolytic enzyme can be used in the present invention. Enzyme Nomenclature, 1992, Webb Academic Press, Inc, N.Y. § EC3.2. It will be recognized by one skilled in the art that other hydrolases can be used in the present invention.

Proteolytic Enzyme

One class of hydrolases is proteolytic enzymes, which are enzymes that hydrolyze complex proteins into simpler soluble proteins. Contemplated proteolytic enzymes for use in the invention include papain, bromelain, protease, serrapeptase, chymotrypsin, trypsin, fungal protease, amylase, plasmin, fibrinolysin, or snake venom protease can be used in the present invention. It will be recognized by one skilled in the art that other proteolytic enzymes can be used in the present invention. Enzyme Nomenclature, 1992, Webb Academic Press, Inc., N.Y. § EC3.4.

Carbohydrate-based Gel Polymer

Gels are defined as semisolid systems consisting of dispersions made up of either small inorganic particles or large organic molecules enclosing and interpenetrated by a liquid. Ansel, H. C., et al., 1999, Pharmaceutical dosage forms and drug delivery systems, 7th ed., Lippincott Williams & Wilkins, Philadelphia, pp 375-381. In the cosmetic industry, the term “gel” is used to describe semi-liquid products. Schueller, R. & Romanowski, P., 1999, Beginning Cosmetic Chemistry, Allured Publishing Corporation, Carol Stream, Ill., p. 99. Depending on the formulation and preparation method, materials ranging from viscous liquids, soft gels, and tough films can be obtained with widely varying gel formation kinetics.

Desirable gels may be formed using carbohydrate polymer as the gelling agent. Polymers preferred for use in the invention include carbohydrate polymers such as the dextrans, dextrins, alginates, starches, modified and fractionated starches, glycogen, pullullan, agarose, cellulose, chitosan, carrageenan, carboxymethylcellulose, alginate, pectin, amylopectin, and synthetic biopolymers, as well as naturally-occurring, synthetic or semi-synthetic gums such as xanthan gum, guar gum, locust bean gum, gum arabic, tragacanth gum, and karaya gum, derivatives thereof and mixtures thereof. These carbohydrate polymers, many of which are classified as polysaccharides or oligosaccharides or derivatives thereof, have the desirable properties of being inert to biological systems. Additionally, they are well characterized and nontoxic, and, due to their water solubility and dispersion characteristics, they may readily be administered in aqueous compositions. Food Chemistry, 3rd Edition. Owen R Fennema, ed., Marcel Dekker, Inc., N.Y., N.Y., 1996, p124-128; Ansel, H. C., et al., 1999, Pharmaceutical dosage forms and drug delivery systems, 7th ed., Lippincott Williams and Wilkins, Philadelphia, pp 375-381.

Other Additives

Embodiments of the composition may also contain conventional additives suitable for improving its properties and characteristics. These may be selected from preservatives, aromatizing agents, humectants and mixtures thereof.

The cosmetic benefits of this invention are improved skin tone, softer, smoother skin, diminished fine lines and wrinkles and an improved appearance of the skin.

After topical application, the composition may be left on the skin, or it may be removed with soap and water. The quantity and extent of the application will vary with the particular result desired, depending on the severity and the condition to be treated, the subject's skin sensitivity, the duration of the treatment, and the nature of concurrent therapy. It is well within the purview of the skilled artisan to regulate dosages according to the subject's need. The composition is directly applied to the involved areas of the skin once per day, twice per day, or three times per day. The composition is applied at least once a week, preferably 2, 3, 4, 5, 6 or 7 times a week. Alternatively, the composition may be applied directly to the skin less frequently or only to achieve certain of the benefits described herein. An amount of composition sufficient to significantly induce a positive modification in the condition to be treated, but low enough to avoid serious side effects.

Another preferred mode of administering is chronic administration. “Chronic” administration, as used herein, means that the period of topical application may be over the subject's lifetime, preferably for a period of at least about one month, more preferably from about three months to about twenty years, more preferably from about six months to about ten years, more preferably still from about one year to about five years, thereby resulting in the reduction or prevention of skin wrinkles.

### BRIEF DESCRIPTION OF THE FIGURES

FIG. 1 comprises photos illustrating the reduction of wrinkles after topical application of the composition prepared in Example 1 and employed in Example 2 of the specification.

### EXAMPLES

The following examples are intended to illustrate specific embodiments of the invention. They are not intended to limit the invention in any manner.

### EXAMPLE 1

### Preparation of Composition

A skin treatment formulation in accordance with the present invention having the following composition was prepared as set forth below:

| Ingredient | % by weight |
|---|---|
| Papain | 5-25 |
| Urea | 5-25 |
| Boric acid | 0.5-10 |
| Methylparaben | 0.15-0.4 |
| Propylparaben | 0.1-0.3 |
| 2-pyrrolidone-5-carboxylic acid, sodium salt | 1-6 |
| Almond extract | 0.01-0.1 |
| Distilled water | 10-75 |
| Amaze XT ™ (xanthan gum) | 1-5 |

Procedure: The composition was prepared by mixing the ingredients: papain, urea, boric acid, methylparaben, propylparaben, sodium salt of 2-pyrrolidone-5-carboxylic acid, Amaze XT™ (xanthan gum), and almond extract. The final pH should be about 5 to 7. Mixing was continued until a uniform mixture was obtained.

### EXAMPLE 2

### Wrinkle Reduction Treatment Protocol

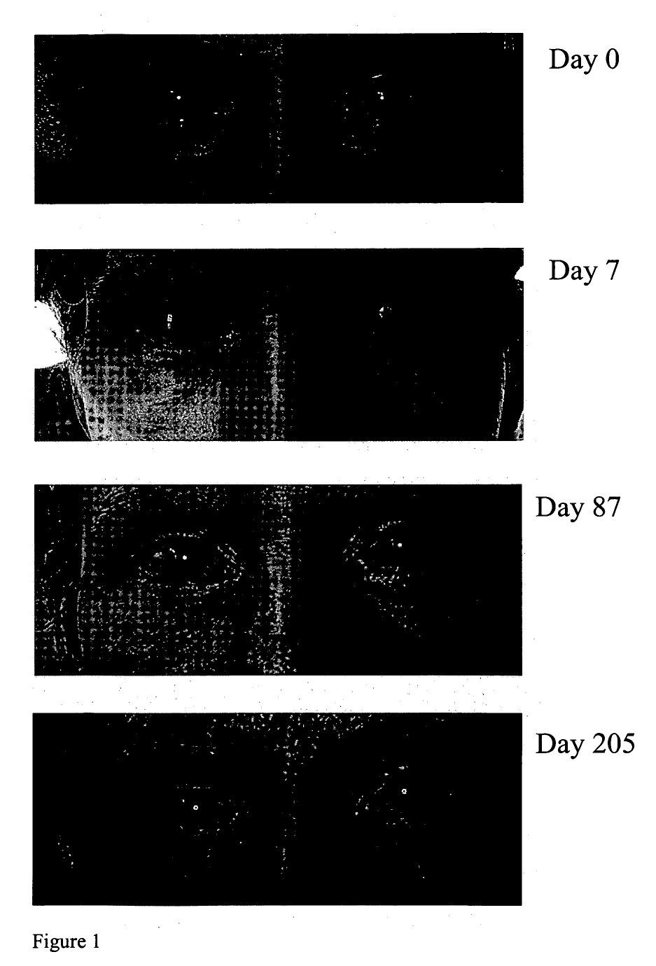

In this example, the composition demonstrated reduction of wrinkles on the subject (FIG. 1). An experiment was performed to screen the potential for the composition to reduce skin wrinkles. A subject with the involved area being the wrinkles below the eye was treated 1-3 times a day, 5-7 days a week for 205 days by applying for 30 seconds to 2 hours a cotton ball, cotton pad, or cotton swab moistened with the composition of Example 1. The area below the patient's eyes was treated. The results show that the treated skin area was more soft and elastic with a smother texture, and the number and depth of the wrinkles decreased. This experiment established that the topical application of the composition of the present invention resulted in improved luminosity, moisturization, satinity and elasticity of the skin. Visible signs of skin aging in the involved area, i.e. the depth of the wrinkles and fine lines, was reduced. The present invention was also tested on eight other subjects, and similar results were obtained.

### EXAMPLE 3

### Nonwoven Wet Wipes

Another aspect of the invention is directed to nonwoven wet wipes which are impregnated or pre-moistened with an effective amount of the composition of the invention.

As used herein, the term “wet wipe” is meant to refer to a non-woven web of fibers that can be used for cleansing purposes. The wet wipe can also be referred to as a towelette.

Wet wipes for topical application of a composition to the skin are well known in the art. Numerous wet wipe materials and packaging have been described in the art, e.g. U.S. Pat. Nos. 6,440,437; 5,629,081; 5,629,081; 6,028,018; 6,440,437; 5,888,524; 5,540,332; 6,673,358; 6,613,729; 6,083,854, all incorporated by reference. These tissues may be prepared from paper or paper products which are treated in such a manner so as to improve their wet strength. Such tissues are generally sized such that they are useful for use as a cloth for application to the skin. They may be packaged in closed containers. Alternatively, the tissues may be individually packaged.

Embodiments of wet wipes are formed from a single layer of a substantially homogeneous material. For example, conventional wet wipes have included an air laid web of fibers which are uniformly mixed or distributed throughout the web. By way of example, not by limitation, wipes have included polymeric fibers such as polyester, polyethylene and polypropylene and natural or synthetic fibers such as cellulosic fibers. Other conventional wet wipes have included a coformed web polypropylene and cellulosic fibers wherein the fibers are uniformly mixed throughout the web. Other embodiments of wet wipes comprise multiple layers of material, i.e. multilaminate. The wet wipe is fabricated from any suitable combination of materials and components known in the art for desired wiping or skin topical application properties. The components may include water, emollients, surfactants, fragrances, preservatives, chelating agents, pH buffers or combinations thereof as are well known to those skilled in the art. The liquid may also contain lotions and/or medicaments.

The amount of the liquid composition in the wet wipe of the invention involves an amount effective for reducing or preventing wrinkles, which in a method of the invention involves the step of contacting the subject skin with the wet wipe. That amount contained within each wet wipe may vary depending upon the type of material being used to provide the wet wipe, the type of liquid being used, the type of container being used to store the wet wipes, and the desired end use of the wet wipe. Methods of fabricating, moistening, and packaging wet wipes are well known in the art.

An embodiment of the invention includes strips of wet wipes made from a nonwoven rayon/polyester blend and impregnated with an effective amount of the compositions of the invention. In one aspect, the wet wipe is impregnated with either a gel-version of the composition, or a non-gel version. The strips, which may be any desired shape, are packaged in a jar for consumer use. In a method of the invention, the consumer applies or contacts a wrinkled section of the face with a strip of the wet wipe. Preferably, the wet wipe is allowed to remain in place, and may be removed from the face any time up to and included when the strip has dried.

Further embodiments of the wet wipe of the invention include strips of wet wipe having one side covered with a flexible, semi- or non-permeable plastic sheet, i.e. a barrier layer. This embodiments slows or stops evaporation of water from the wet wipe, which allows the composition of the invention impregnated in the strip to remain on the skin for a longer period. Other embodiments involve wet wipes, fabricated with semi- or non-permeable plastic sheets, as above, and further comprising adhesive positioned on one or more borders of the strip for contact and adherence to the skin over which the strip is placed.

A further method of the invention involves the step of applying the composition of the invention to a subject facial area, and an additional step of positioning an adhesive or non-adhesive wet wipe over the applied composition. In this instance, the wet wipe does not necessarily comprise a composition of the invention.

While the invention has been described by reference to specific embodiments, this is for illustrative purposes only. Various modifications to the above invention will become apparent to those skilled in the art, all of which are intended to fall within the spirit and scope of the present invention. All patents and publications referred to herein are hereby incorporated by reference.

What is claimed is:

1. A wet wipe comprising an effective amount of an aqueous composition for removing and preventing wrinkles, comprising:
a. an aqueous composition comprising a hydrolase
b. a wipe comprising a semi-or non-permeable barrier layer.

2. (canceled)

3. (canceled)

4. (canceled)

5. The wet wipe of claim 1 wherein the hydrolase is selected from the group consisting of carboxyesterases, triacylglycerol lipases, esterase, lipoprotein lipases, or proteolytic enzymes.

6. The wet wipe of claim 5 wherein the proteolytic enzyme is selected from the group consisting of papain, bromelain, protease, serrapeptase, chymotrypsin, trypsin, fungal protease, amylase, plasmin, fibrinolysin, ficin, snake venom proteases, and peptidase.

7. The wet wipe of claim 1 comprising papain enzyme in an amount of from about 5 to 25% by weight; about 5 to 25% by weight of urea; about 0.5 to 10% by weight of boric acid; about 0.15 to 0.4% by weight of methylparaben; about 0.1 to 0.3% by weight of propylparaben; about 10 to 80% distilled water; about 1 to 6% by weight of the sodium salt of 2-pyrrolidone-5-carboxylic acid; and about 0.01 to 0.1% by weight of almond extract.

8. (canceled)

9. The wet wipe of claim 7 wherein the pH is about 5 to 7.

10. (canceled)

11. The wet wipe of claim 7 wherein the papain, urea, boric acid, methylparaben, propylparaben, distilled water, sodium salt of 2-pyrrolidone-5-carboxylic acid, and almond extract are blended to form a mixture.

12. (canceled)

13. (canceled)

14. (canceled)

15. (canceled)

16. A method for making a wet wipe, the method involving the step of impregnating a wet wipe with an effective amount of an aqueous composition for removing and preventing wrinkles, the composition comprising a hydrolase.

17. An aqueous composition comprising papain enzyme in an amount of from about 5 to 25% by weight; about 5 to 25% by weight of urea; about 0.5 to 10% by weight of boric acid; about 0.15 to 0.4% by weight of methylparaben; about 0.1 to 0.3% by weight of propylparaben; about 10 to 80% distilled water; about 1 to 6% by weight of the sodium salt of 2-pyrrolidone-5-carboxylic acid; and about 0.01 to 0.1% by weight of almond extract.
